(19) United States
(12) Patent Application Publication
(10) Pub. No.: US 2006/0058779 A1
(43) Pub. Date: Mar. 16, 2006

(54) Electrocautery method and system

(76) Inventor: Maryam Broukhim, Roslyn Hardor, NY (US)

Correspondence Address: STROOCK & STROOCK & LAVAN LLP, 180 MAIDEN LANE, NEW YORK, NY, 10038, US

(21) Appl. No.: 10/536,203

(22) PCT Filed: Jul. 15, 2004
(86) PCT No.: PCT/US04/22688

§ 371 Date: May 24, 2005

Related U.S. Application Data
(60) Provisional application No. 60/488,179, filed on Jul. 17, 2003.

(51) Int. Cl. A61B 18/04

(52) U.S. Cl. 606/27

(57) ABSTRACT

A system and method for performing electrocautery is provided in which the surgeon need not worry about injuring the patient by placing the electrocauteral blade too close to the retractor. This system and method is implemented by forming at least the retracting end of the retractor from an electrically non-conductive material. In one embodiment of the invention, the retractor is formed from the electrically insulating material, such, for example, plastic, especially heat resistant clear plastic. Thermosetting resins, and other polymers particularly those having filters to increase heat resistance are preferred. The electrically non-conductive retractor is used to retract the skin as the electrocauteral blade is used to cut and/or coagulate. Methods in accordance with invention are particularly useful when the initial incision is small, such as in the one to five, preferably two to four inch range. Methods and systems in accordance with the invention are particularly preferred during cosmetic surgery and/or surgery on the breasts, head or neck. Accordingly, it is an object of the invention to provide an improved system and method for performing electrocautery.

### CROSS-REFERENCE TO RELATED APPLICATIONS

This application claims the benefit of Provisional Patent Application No. 60/488,179, which is incorporated herein by reference.

### BACKGROUND OF INVENTION

The invention relates to a system for performing electrocauteral surgery (i.e., electrosurgery) that permits more precise use of the electrocauteral blade and helps prevent scarring and discoloration of the skin and other body tissue during electrocautery.

Electrocautery involves the application of relatively high-frequency or radio frequency (RF) current to living tissue. By varying the characteristics of the current, an electrocauteral apparatus can be used to cut tissue, to coagulate bleeding from the tissue (hemostasis) or to both cut tissue and promote hemostasis simultaneously. One example of an electrocauteral device is the Valleylab Force Fx Instant Response Electrosurgical Generator, sold by Valleylab Inc. of Boulder, Colo.

Electrocautery is performed by connecting the electrocauteral blade or active electrode to an electrocauteral generator, activating the generator to supply the electrocauteral waveform, and delivering the energy of the electrocauteral waveform to the tissue through the blade. The blade is typically positioned in a pencil-like hand piece, which the surgeon manipulates to achieve the desired effect at the surgical site.

Cutting is achieved by bringing the leading edge into close adjacency with the tissue. A high current density at the narrow leading edge transfers energy into the tissue as relatively short arcs, thereby causing enough heat to explode or rupture the cells of the tissue at the interface with the narrow leading edge. The tissue separates at the leading edge leaving a well-defined incision. It is in this manner that the current from the electrocauteral blade cuts the tissue, rather than the tissue being separated from the physical contact and mechanical action of a sharp edge, as is the case with a traditional scalpel. In fact, the narrow edge of the typical electrocauteral blade is typically not sharp and typically cannot cut tissue as a result of mechanical action. The separated tissue passes by the broad sides of the working area of the active electrode as the surgeon guides the blade, while the electrical energy creates the incision.

Coagulating bleeding surfaces usually involves bringing the tip of the working area of the blade to a point spaced slightly above the bleeding surface and delivering a duty cycle type of coagulating electrocauteral waveform.

Typically, as electrocautery is performed, the tissue is pulled back by use of a retractor. Retractors come in various shapes and configurations. For example, common retractors, such as Kelly or Richardson retractors available from the Allegiance Healthcare Company, Dearfield, Ill., are generally “J” or “L” shaped and are formed with a handle, a handle extension leading to the bottom of the “L” or “J”. The distal end of the retractor is generally flattened curved. The distal end is commonly convex when looking at the curved portion from the inside of the curve.

Retractors are conventionally formed from steel, such as stainless steel, which is easy to sterilize and can be quite strong. Frequently, considerable force is needed to pull tissue out of the way during electrocautery.

Care must be taken to ensure that electrocauteral blade does not come too close to a conventional retractor. For example, arcing of current from the electrocauteral blade from the retractor can occur if the blade is positioned too close to the retractor. This can lead to burning or discoloration of the tissue or skin in contact with the retractor. This can be particularly undesirable during certain surgeries, such as breast, head or neck surgery, as undo trauma, discoloration or otherwise altering the skin in the breast area can be deleterious to the patient's psychological condition as well as leading to medical complications.

Accordingly, it is desirable to provide a system for performing electrocautery, which overcomes problems associated with arcing and burning, which can occur when electricity is conducted into the retractor.

### SUMMARY OF THE INVENTION

Generally speaking, in accordance with the invention, a system and method for performing electrocautery is provided in which the surgeon has less concern about injuring the patient by placing the electrocauteral blade too close to the retractor. This system and method is implemented by forming the retractor from a non-electrically conductive material. In one embodiment of the invention, the retractor is formed from electrically insulating material, such as for example, plastic or plastic-like materials, especially heat resistant clear plastic. The retractor is constructed to be strong enough to retract tissue during electrocauteral surgery. Thermosetting resins, particularly those having fillers or other additives to increase heat or fire resistance are preferred. The non-conductive retractor is used to retract the skin as the electrocauteral blade is used to cut and/or coagulate. Methods in accordance with invention are particularly useful when the initial incision is small, such as in the one to five, preferably two to four inch range. Methods and systems in accordance with the invention are particularly preferred during cosmetic surgery and/or surgery on the breasts, head or neck.

Accordingly, it is an object of the invention to provide an improved system and method for performing electrocautery.

### BRIEF DESCRIPTION OF THE DRAWINGS

For a fuller understanding of the invention, reference is had to the following description taken in connection with the accompanying drawing, in which:

FIG. 1 is a perspective view of a retractor in accordance with one embodiment of the invention.

### DETAILED DESCRIPTION OF THE INVENTION

The invention relates to a system for performing electrocautery that permits more precise use of the electrocauteral blade and helps prevent scarring and discoloration of the skin during electrocautery. Systems in accordance with the invention comprise an electrocauteral apparatus in combination with an electrically non-conductive retractor.

The electrocautery is performed by the application of relatively high-frequency or radio frequency (RF) current to living tissue. By varying the characteristics of the current, the electrocauteral apparatus can be used to cut tissue, to coagulated bleeding from the tissue or to both cut tissue and promote hemostasis simultaneously. Acceptable frequencies of the electrocauteral device can range from approximately 400 kHz to 750 kHz. The electrical power applied can vary from a few watts for delicate neurosurgical procedures to 300 watts or more for cutting substantial tissues in open surgical procedures. The open circuit voltage prior to imaging transfer to the tissue can be in the range of 5,000-10,000 volts peak to peak. The voltage will typically drop substantially as the current flow increases as a result of the impedance of the tissue. Typical tissues impedances range from about 10 to 500 ohms.

One example of an electrocauteral device is the Valleylab Force Fx Instant Response Electrosurgical Generator, sold by Valleylab Inc. of Boulder, Colo.

Electrocautery is performed by connecting the electrocauteral blade or active electrode to an electrocauteral generator, activating the generator to supply the electrocauteral waveform, and delivering the energy of the electrocauteral waveform to the tissue through the blade. The blade is typically positioned in a pencil-like hand piece, which the surgeon manipulates to achieve the desired effect at the surgical site. Selecting and adjusting the characteristics of the electrocauteral waveform delivered by the electrocauteral generator allows the surgeon to cut the tissue, to coagulate bleeding from the tissue, or to simultaneously cut and coagulate. The ability to control the application of electrical energy to the tissue to cut and coagulate tissue is one of the substantial advantages of electrocautery.

The physical characteristics of the electrocauteral blade can be used advantageously by the surgeon to accomplish different surgical procedures. An acceptable electrocauteral blade has an elongated working area with a shape similar to a rectangle in cross-section. Two relatively broad and generally parallel sides extend along and exist on opposite sides of the working area. The two broad sides are joined by a narrow edge which extends between the broad sides and which curves around a distal end or tip of the working area. The edges form the narrow legs of the cross-sectional rectangle while the broad sides form the wide legs of the cross-sectional rectangle.

Cutting can be achieved by bringing the narrow edge into close adjacency with the tissue to be cut. A high current density at the narrow leading edge transfers energy into the tissue as relatively short arcs, thereby causing enough heat to explode or rupture the cells of the tissue at the interface with the narrow leading edge. The tissue can separate at the leading edge leaving a well-defined incision. The separated tissue passes by the broad sides of the working area of the active electrode as the surgeon guides the blade, while the electrical energy creates the incision.

Coagulating bleeding surfaces can involve bringing the tip of the working area of the blade to a point spaced slightly above the bleeding surface and delivering a duty cycle type of coagulating electrocauteral waveform. The duty cycle coagulating waveform can include an on time period during which the high frequency electrical signal is delivered, followed by an off-time during which no electrical energy is delivered. The coagulating duty cycles can be repeated at a frequency in the neighborhood of approximately 30 kHz, with a power of approximately 50-80 watts. Longer arcs of electrical energy are transferred in a spray-like manner from the tip of the blade and these arcs penetrate into the tissue to create a reticulum, which can both activate the normal clotting mechanism in the blood and thermally seal the surface of the tissue. Bleeding vessels can be coagulated in much the same manner except that the tip of the blade is sometimes placed in close adjacency with the severed vessel, causing the arcs to be concentrated at that location.

Simultaneous cutting and coagulating can be performed by blending the duty cycle coagulation waveform with a continuous waveform. This can involve increasing the on-time of the duty cycle to sufficient amount, which permits cutting to occur, but which still allows coagulation to be achieved. Because of the relative convenience and quickness with which coagulation can be achieved, many surgical procedures can progress more rapidly by using electrocautery than if electrocautery was not used.

The amount of electrical energy delivered to the tissue through the electrocauteral blade is typically high and the current flow through the blade itself heats the blade. Since the typical tissue impedance ranges in the neighborhood of tens of ohms to a few hundreds of ohms, the impedance of the blade itself is significant enough relative to the impedance of the tissue that the blade absorbs enough of the transferred energy to increase its temperature significantly during electrocautery.

As electrocautery is performed, the tissue is pulled back by use of a retractor. Various configurations are acceptable. Retractors in accordance with the invention can be formed with a handle, a handle extension and a flattened curved portion which is commonly convex when looking at the curved portion from the inside of the curve.

Retractors in accordance with invention are strong and are electrically insulating at least their distal ends that come into close proximity with the electrocauteral blade.

In one embodiment of the invention, the retractor is formed of steel, preferably stainless steel or other metal and is covered with an insulating material. Examples of insulating covering include ceramic material, glazed ceramic material, porcelain, natural and synthetic polymers, certain configuration of rubber, silicone, plastic and resin. Preferably the coating is heat resistant. It also preferably contains fire retardant materials.

In another more preferred embodiment of the invention, the retractor is formed of electrically insulating material, at least at the working end, or in its entirety. It can be formed of plastic or plastic like materials Preferably, the plastic or plastic-like material is biocompatible. The plastic should also be resistant to heat and should include fire proof or fire retardant materials. Polymers, resins, thermosetting resins and epoxies are preferred. Generally, plastics in accordance with the invention will include filler, such as ceramic particles or silica and the like, which can increase the heat resistant properties thereof. Electrically conductive filler materials should be avoided.

In another embodiment of the invention, the retractor or at least the distal end portion thereof is transparent. In this manner, the surgeon can look through the retractor and observe the tissue located behind the retractor and thereby improve their performance of the surgical procedure.

Retractors in accordance with the invention are typically J-shaped or L-shaped, with a proximal handle end and a curved distal “working” end. The curved end includes a leading lip, which is joined to the handle portion with a curved portion and then an extension portion. Preferably, the leading lip and curved portion are not electrically conductive. The extension portion and handle portion can also be made of electrically insulating material. The working end is preferably continuous from edge to edge to achieve proper retraction of the tissue.

In one embodiment of the invention, the curved portion includes an inner curved surface, which is substantially flat and a rim, which descends away from and is substantially perpendicular from the flat inner curve surface.

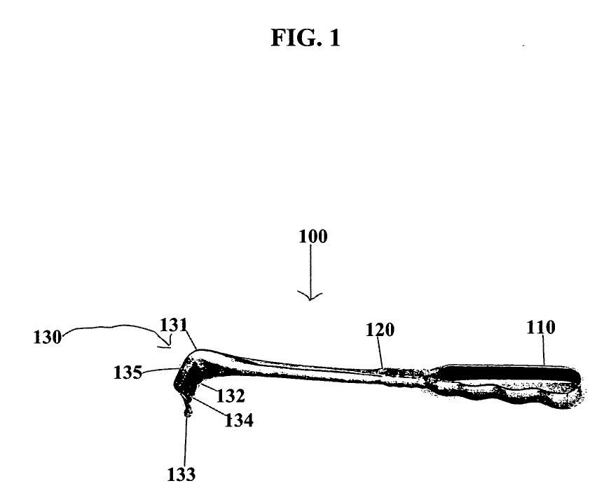

An acceptable shape for retractors formed in accordance with the invention is shown in FIG. 1, which is not intended to be construed in a limiting sense. A retractor for use in accordance with the invention is shown generally as retractor 100 in FIG. 1. Retractor 100 includes a handle 110, a handle extension 120 and a working end 130. Working end 130 includes a curved portion 131 transitioning from handle 120, a distal tip 133 and a working surface 132 extending from curved portion 131 to distal tip 133. Distal tip 133 is also deflected inwards at a bend 134. The thickness of working end 130 is fairly uniform. Working end 130 also includes a descending ledge 135 on both sides thereof, which descend away from the inner top surface of working portion 132.

Accordingly, in view of the insulating properties of retractors in accordance with the invention, electrocautery can be performed at a higher power setting than would ordinarily be employed, because there is less chance of problems associated with arcing or other contact between the electrocauteral device and the retractor. The blade can be brought within 1-3 millimeters of, or even touch, the retractor without adverse complications.

It will thus be seen that the objects set forth above, among those made apparent from the preceding description, are efficiently attained and, since certain changes may be made in carrying out the above method and in the article set forth without departing from the spirit and scope of the invention, it is intended that all matter contained in the above description and shown in the accompanying drawing shall be interpreted as illustrative and not in a limiting sense.

What is claimed is:

1. A method for performing breast surgery, comprising: using an electrocauteral device having a cutting tip to cut skin and other tissues and making an incision of about 1 to 5 inches and delivering an effective amount of electrical current at an effective voltage and waveform to cauterize the area of the cut; at the same time, using a retractor with an electrically non-conductive retracting end to retract the tissue at the areas of the incision in close proximity to the cutting tip, the retracting end being strong enough and configured to retract tissue at the area of the cut during electrocauteral surgery.

2. The method according to claim 1, wherein the retractor comprises metal, covered at least in part with an electrically insulating material.

3. The method according to claim 2, wherein the metal comprises steel or stainless steel.

4. The method according to claim 2, wherein the insulating material is selected from the group consisting of ceramic material, glazed ceramic material, porcelain, rubber, silicone, plastic, plastic-like materials, epoxies, resins and other polymeric materials.

5. The method according to claim 2, wherein the insulating covering comprises a fire retardant material.

6. The method according to claim 1, wherein the retractor is formed substantially entirely of electrically insulating material.

7. The method according to claim 6, wherein the insulating material is heat resistant and selected from the group consisting of ceramic material, glazed ceramic material, porcelain, rubber, silicone, polymers, epoxy and resin.

8. The method according to claim 6, wherein the insulating material further comprises heat resistant and fire retardant components.

9. The method according to claim 6, wherein at least a portion of the retracting end is substantially transparent.

10. The method according to claim 1, wherein the insulating material comprises plastic or plastic-like material.

11. An electrocauteral surgery system comprising: an electrocauteral device comprising a cutting tip designed to deliver a cauterizing amount of electricity to the tip; and a retractor formed with a retracting end having a hook-like portion comprising electrically insulating material.

12. The system according to claim 11, wherein the retractor comprises metal covered with electrically insulating material.

13. The system according to claim 12, wherein the metal comprises steel or stainless steel.

14. The system according to claim 12, wherein the insulating material is selected from the group consisting of ceramic material, glazed ceramic material, porcelain, rubber, silicone, plastic, epoxy and resin.

15. The system according to claim 12, wherein the insulating material is heat-resistant and comprises fire retardant additives.

16. The system according to claim 11, wherein the retractor is formed substantially entirely of electrically insulating material.

17. The system according to claim 11, wherein the insulating material is selected from the group consisting of ceramic material, glazed ceramic material, porcelain, rubber, silicone, plastic, epoxies and resins.

18. The system according to claim 11, wherein the insulating material comprises plastic or plastic-like materials and is sufficiently strong to retract tissue during electrocauteral surgery.

19. The system according to claim 11, wherein the insulating material is transparent.
